(19) United States
(12) Patent Application Publication
(10) Pub. No.: US 2003/0161757 A1
(43) Pub. Date: Aug. 28, 2003

(54) Method of sterilizing mail

(76) Inventor: Charles E. Evans, Pueblo, CO (US)

Correspondence Address: Glen F. Gallinger, 7420 Milner Dr., #1000, Colorado Springs, CO, 80920, US

(21) Appl. No.: 10/084,076

(22) Filed: Feb. 27, 2002

(51) Int. Cl. A61L 2/08

(52) U.S. Cl. 422/22; 422/24; 232/17; 250/455.11

(57) ABSTRACT

A method of automatically sterilizing mail all mail received provides assurance to the recipients of the mail and facilitates efficient processing of the mail. The method comprises the steps of: providing an internal electromagnetic radiation source to sterilize mail deposited in the mail box; providing an electric door switch serially connected to the electromagnetic radiation source adapted to close only when the mail box door is closed so that the electromagnetic radiation radiates from the source only when the front door is closed; and, providing a sterilization timer activated when the mail box door is closed, said timer configured to maintain the electromagnetic source on for a sufficient period after the mail box door is closed to radiate and thereby sterilize any mail deposited therein. One aspect of the invention comprises a method of retrofitting a mail box with an assembly containing all of the components listed above.

### FIELD OF THE INVENTION

This invention relates to contamination of mail by infectious microbes. More particularly this invention relates to a mail box which automatically sterilizes all mail received therein.

### BACKGROUND OF THE INVENTION

Recent events have underscored the susceptibility of the American public to contaminated mail. Terrorists or other individuals can deposit contaminated mail targeted to a specific individual in any one of almost 100 million mail box drops or delivery boxes. The probability of a culpable individual being impugned or being otherwise apprehended is almost non-existent. The effect on the targeted individual may be capital. And the effects on millions of non-targeted individuals may be paralyzing.

What is needed is a sterilizing mail box. A sterilizing mail box would provide its owners and recipients of mail deposited therein with assurance that they would not be susceptible to contaminated mail.

### OBJECTS OF THE INVENTION

It is an object of this invention to disclose a convenient method of sterilizing all mail received. It is an object of this invention to disclose an automated method of sterilizing all mail received so that all mail received can subsequently be expeditiously opened, without any fear of contamination or infection. It is yet a further object of this invention to disclose a convenient method of retrofitting any existing mail box with an assembly which will automatically sterilize all mail deposited therein. It is a final object of this invention to disclose a method of sterilizing a high volume of mail using a mail box fitted with a microwave source of electromagnetic radiation.

One aspect of this invention provides for a method of sterilizing mail received in a mail box having a front door comprising the steps of: providing an internal electromagnetic radiation source to sterilize mail deposited in the mail box; providing an electric door switch serially connected to the electromagnetic radiation source adapted to close only when the mail box door is closed so that the electromagnetic radiation radiates from the source only when the front door is closed; and, providing a sterilization timer activated when the mail box door is closed, said timer configured to maintain the electromagnetic source on for a sufficient period after the mail box door is closed to radiate and thereby sterilize any mail deposited in the mail box.

A preferred aspect of this invention comprises a method as above further comprising the step of providing an assembly having an anti-microbial florescent lamp as the source of electromagnetic radiation. A housing of the florescent lamp is adapted to house a push button electric door switch and the sterilization timer. The preferred method further comprises the step of positioning florescent lamp tube in an upper portion of the mail box so that the electric switch therein is closed only when depressed by the closed front door of the mail box.

Various other objects, advantages and features of this invention will become apparent to those skilled in the art from the following description in conjunction with the accompanying drawings. FIGURES OF THE INVENTION

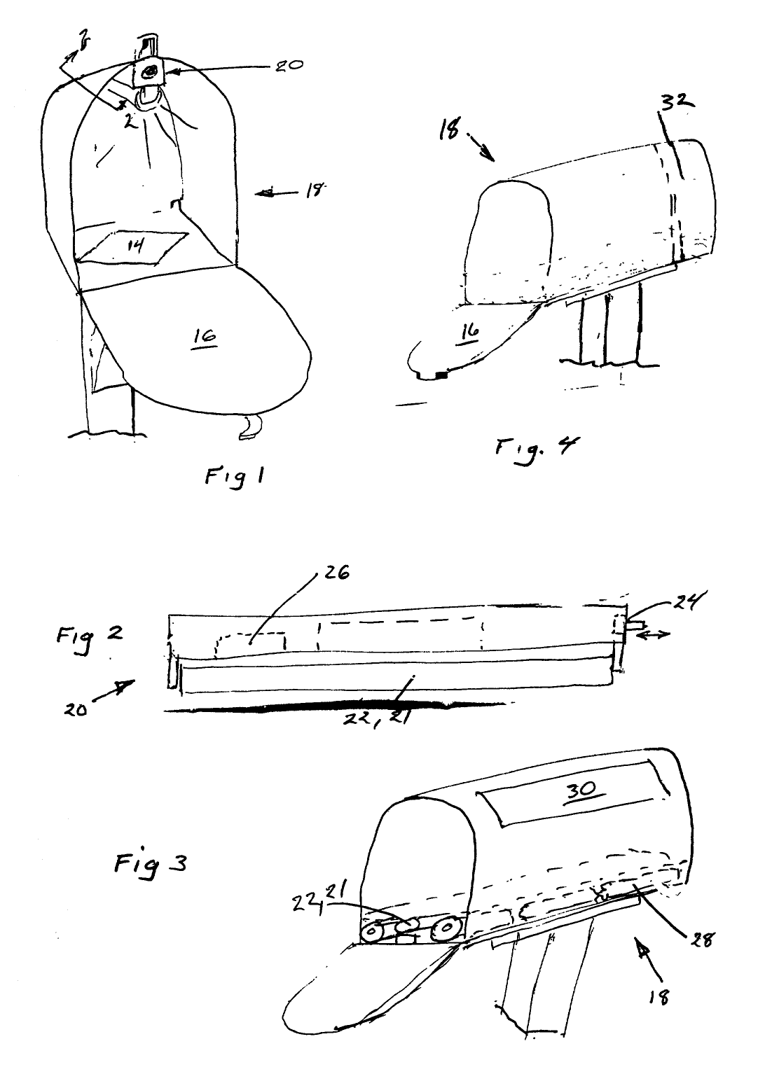

FIG. 1 is a perspective view of a mail box retrofitted with an ultra violet light apparatus to automatically sterilize all mail deposited therein.

FIG. 2 is an enlarged side perspective view of the retrofitted assembly as viewed along line 2-2 in FIG. 1.

FIG. 3 is a perspective view of a mail box having an ultra violet light and battery power pack in its lower portion, as well as a photo cell on an upper exterior portion thereof.

FIG. 4 is an alternative embodiment of the invention comprising a mail box adapted for microwave sterilization of mail.

The following is a discussion and description of the preferred specific embodiments of this invention, such being made with reference to the drawings, wherein the same reference numerals are used to indicate the same or similar parts and/or structure. It should be noted that such discussion and description is not meant to unduly limit the scope of the invention.

### DESCRIPTION OF THE INVENTION

Turning now to the drawings and more particularly to FIG. 1 we have perspective view of a mail box 18 retrofitted with an ultra violet light assembly 20 to automatically sterilize all mail 16 deposited therein. FIG. 2 is an enlarged side perspective view of the retrofitted assembly 20 as viewed along line 2-2 in FIG. 1. The retrofitted assembly 20 is a means of providing an internal electromagnetic radiation source 22 to sterilize mail 16 deposited in the mail box 18. The retrofitted assembly 20 comprises an electric door switch 24 serially connected to the electro magnetic radiation source 22. The switch 24 is adapted to close only when the mail box door 16 is closed so that the electromagnetic radiation radiates from the source 22 only when the front door 16 is closed. A sterilization timer 26 is activated when the mail box door 16 is closed. The sterilization timer is configured to maintain the electromagnetic source 22 on for a sufficient period after the mail box door 16 is closed to radiate and thereby sterilize any mail 14 deposited in the mail box 18. Most preferably the ultraviolet radiation source 22 comprises a florescent tube 21 adapted to be positioned within the interior of the mail box 18.

FIG. 3 is a perspective view of a mail box 18 having an ultra violet radiation source 22 and battery power pack 28 in its lower portion to power the radiation source 22. Mail 14, most preferably supported in an upright position, is radiated through a transparent mail floor 34. A rack (not shown) may be provided to facilitate holding the mail 14 in a vertical position so that it may be more fully radiated. The method may additionally include the step of providing a photo cell 30 on an upper exterior portion of the mail box 18. The photo cell 30 charges the battery power source 28. Having a self charging battery 28 facilitates positioning the mail box 18 in a location which is distant from an electric power source (not shown).

FIG. 4 is yet another embodiment of the invention which comprises a mail box 18 adapted for microwave sterilization of mail 14. In this embodiment of the invention the mail box 18, and the mail box door 16 must be metal. A microwave emitter 32 is preferably positioned within a rear portion of the mail box 18.

While the invention has been described with preferred specific embodiments thereof, it will be understood that this description is intended to illustrate and not to limit the scope of the invention, which is defined by the following claims.

I claim:

1) A method of sterilizing mail received in a mail box having a front door comprising the steps of:
providing an internal electromagnetic radiation source to sterilize mail deposited in the mail box;
providing an electric door switch serially connected to the electromagnetic radiation source adapted to close only when the mail box door is closed so that the electromagnetic radiation radiates from the source only when the front door is closed; and,
providing a sterilization timer activated when the mail box door is closed, said timer configured to maintain the electromagnetic source on for a sufficient period after the mail box door is closed to radiate and thereby sterilize any mail deposited in the mail box.

2) A method as in claim 1 wherein the electromagnetic radiation source comprises anti-microbial radiation.

3) A method as in claim 2 wherein the anti-microbial radiation source comprises a florescent lamp, having a florescent tube, adapted to be positioned within the interior of the mail box.

4) A method as in claim 3 further comprising the step of providing an assembly comprising the florescent lamp having a housing adapted to hold the florescent tube, said housing additionally housing the sterilization timer and the electric switch in an end portion of the housing; and wherein the electric switch is a push button switch which closes when it is depressed.

5) A method as in claim 4 further comprising the step retrofitting a separately purchased mailbox by mounting and positioning the assembly in an upper portion of the mail box so that the electric switch is adjacent to and depressed only when the front door of the mail box is closed.

6) A method as in claim 2 further comprising a mail box and a battery power pack for the ultraviolet radiation source.

7) A method as in claim 6 further comprising the step of providing a photo cell on the upper exterior of the mail box configured to charge the battery power pack.

8) A method as in 2 wherein the florescent tube is positioned within a lower portion of the mail box and is covered by a transparent mail floor.

9) A method as in claim 1 wherein the electromagnetic source comprises a microwave emitter and further comprising a metal mail box having a metal door to contain microwave radiation.

10) A method as in claim 9 wherein the microwave emitter is positioned within a rear portion of the mail box.

11) An assembly for retrofitting a mail box having a front door, to sterilize mail received therein, comprising:
a florescent lamp having a housing adapted to hold a florescent tube;
a sterilization timer contained within the housing; and, an electric push button switch positioned within, and projecting through an end portion of the housing;
so that when the assembly is mounted within an upper portion of the mail box, the button portion of the switch is pushed when the mail box door closed, thereby activating the florescent lamp to sterilize any mail contained therein.
